(19) United States
(12) Patent Application Publication
(10) Pub. No.: US 2026/0138864 A1
(43) Pub. Date: May 21, 2026

(54) AIRFLOW GENERATING DEVICE AND ITS APPLICATIONS

(71) Applicant: xMEMS Labs, Inc., Santa Clara, CA (US)

(72) Inventors: Thomas Tarter, San Jose, CA (US); Michael David Housholder, San Jose, CA (US); Jye Ren, Taipei City (TW)

(73) Assignee: xMEMS Labs, Inc., Santa Clara, CA (US)

(21) Appl. No.: 19/452,852

(22) Filed: Jan. 19, 2026

Related U.S. Application Data
(63) Continuation-in-part of application No. 18/988,923, filed on Dec. 20, 2024, now Pat. No. 12,546,303.
(60) Provisional application No. 63/618,391, filed on Jan. 8, 2024.
(60) Provisional application No. 63/886,865, filed on Sep. 24, 2025.

(51) Int. Cl. B81B 3/00; B81B 7/00; F04B 45/04

(52) U.S. Cl. CPC B81B 3/0021; B81B 7/0083; F04B 45/043

(57) ABSTRACT

An airflow generating device includes a first flap, a second flap, a first actuator, a second actuator and an anchor structure. The first flap and the second flap are opposite to each other in a top view viewing along a top-view direction. The first actuator is disposed on the first flap, and the second actuator is disposed on the second flap. The first flap includes a first anchored edge anchored on the anchor structure, and the first flap includes first free edges other than the first anchored edge which are non-anchored. The second flap includes a second anchored edge anchored on the anchor structure, and the second flap includes second free edges other than the second anchored edge which are non-anchored.

### CROSS REFERENCE TO RELATED APPLICATIONS

This application is a continuation-in-part of U.S. application Ser. No. 18/988,923, filed on Dec. 20, 2024, which claims the benefit of U.S. Provisional Application No. 63/618,391, filed on Jan. 8, 2024. Further, this application claims the benefit of U.S. Provisional Application No. 63/886,865, filed on Sep. 24, 2025. The contents of these applications are incorporated herein by reference.

### BACKGROUND OF THE INVENTION

### 1. Field of the Invention

The present invention relates to an air quality sensing module and an air pump, and more particularly, to an air quality sensing module and an air pump in a handheld device.

### 2. Description of the Prior Art

As electronic devices undergo continuous miniaturization and performance enhancement, traditional thermal management faces significant challenges. Conventional cooling systems primarily rely on passive heat sinking or rotational mechanical fans. Passive solutions often lack the precision to address concentrated heat generated by localized high-power components, leading to thermal accumulation in confined spaces. Meanwhile, traditional active cooling fans are frequently unsuitable for compact, wearable, or high-density hardware due to their substantial physical volume, audible noise, and mechanical vibrations. Furthermore, the inability of existing systems to provide high-back-pressure airflow within millimeter-scale height constraints often results in device throttling and compromised reliability. Consequently, there remains a critical need for a cooling architecture that provides effective, silent, and localized heat dissipation without the spatial and mechanical drawbacks of conventional methods.

There is a pressing need for improving the prior art.

### SUMMARY OF THE INVENTION

It is therefore a primary objective of the present application to provide an air quality sensing module in a handheld device. Also, the present application further provides a related air pump.

An embodiment of the present application discloses an airflow generating device including a first flap, a second flap, a first actuator, a second actuator and an anchor structure. The first flap and the second flap are opposite to each other in a top view viewing along a top-view direction. The first actuator is disposed on the first flap, and the second actuator is disposed on the second flap. The first flap includes a first anchored edge anchored on the anchor structure, and the first flap includes first free edges other than the first anchored edge which are non-anchored. The second flap includes a second anchored edge anchored on the anchor structure, and the second flap includes second free edges other than the second anchored edge which are non-anchored.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a cross sectional view illustrating an air pump according to an embodiment of the present invention.

FIG. 2 is a schematic diagram of a cross sectional view illustrating a common mode movement and a differential mode movement of the air pump according to an embodiment of the present invention.

FIG. 3 is a schematic diagram of air pulses according to an embodiment of the present invention.

FIG. 4 illustrates waveforms of demodulation signals and a modulation signal according to an embodiment of the present invention.

FIG. 5 is a schematic diagram of an air pump according to an embodiment of the present invention.

FIG. 6 is a schematic diagram of an appearance of a phone according to an embodiment of the present invention.

FIG. 7 is an internal layout diagram of the phone according to an embodiment of the present invention.

FIG. 8 is a (local) detailed view of the internal layout diagram of the phone according to an embodiment of the present invention.

FIG. 9 is a (local) cross-sectional view of the phone according to an embodiment of the present invention.

FIG. 10 is a schematic diagram of a smart glasses according to an embodiment of the present invention.

FIG. 11 is a schematic diagram of a temple structure according to an embodiment of the present invention.

FIG. 12 is a schematic diagram of a memory module according to an embodiment of the present invention.

FIG. 13 is a cross-sectional view of the memory module of FIG. 12.

FIG. 14 is a cross-sectional view of the memory module of FIG. 12.

FIG. 15 is a schematic diagram of a memory module according to an embodiment of the present invention.

FIG. 16 is a schematic diagram of a cross-sectional view of the memory module of FIG. 15.

FIG. 17 is a schematic diagram of an appearance of a pluggable optical transceiver module according to an embodiment of the present invention.

FIG. 18 is a schematic diagram of an exploded view of the pluggable optical transceiver module of FIG. 17.

FIG. 19 is a schematic diagram of an appearance of a circuit board disposed within the pluggable optical transceiver module of FIG. 17.

FIG. 20 is a schematic diagram of a cross-sectional view of the pluggable optical transceiver module of FIG. 17.

FIG. 21 is a schematic diagram of an appearance of a pluggable optical transceiver module according to an embodiment of the present invention.

FIG. 22 is a schematic diagram of an appearance of a cover or a flow guide disposed within the pluggable optical transceiver module of FIG. 21.

### DETAILED DESCRIPTION

In the present invention, the technical features described in the embodiments may be mixed or combined in various ways as long as there are no conflicts between them.

In the present invention, an airflow generating component is configured to generate an airflow, wherein the airflow generating component may be applied in cooling, drying, dehumidifying, heat dissipation, ventilation, air-sampling and/or air-pumping applications by generating the airflow. In the present invention, the airflow generating component may be designed based on requirement(s), and the airflow generating component may be formed by any suitable method. In the following, some embodiments of the airflow generating component are explained.

For example, the airflow generating component may be an air pump or an airflow generating chip, wherein the air pump or airflow generating chip may be formed by a semiconductor manufacturing process. For example, the airflow generating chip may be a micro electro mechanical system (MEMS) chip including a MEMS structure, but not limited thereto.

In the present application, “air pump” and “airflow generating component/chip” refer to the same component and are used interchangeably. Furthermore, the “air pump” and/or “airflow generating component/chip” may realize a fan-on-chip concept, i.e., a component with small size (as small as chip size, where chip length/width can be realized to be less than 15 millimeter (mm)) capable of generating airflow.

Thanks to the small size (as small as chip size with chip length/width less than 15 mm), it is possible to dispose air quality sensing module comprising the airflow generating component/chip in handheld electronic device, and therefore real-time and close-proximity air quality sensing is realizable.

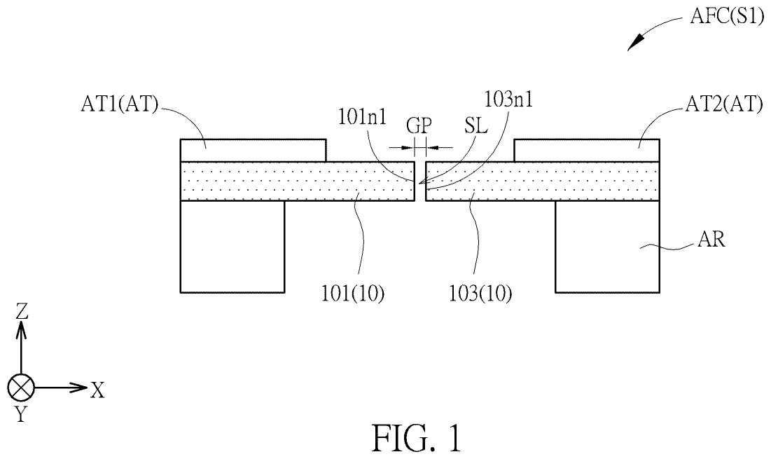

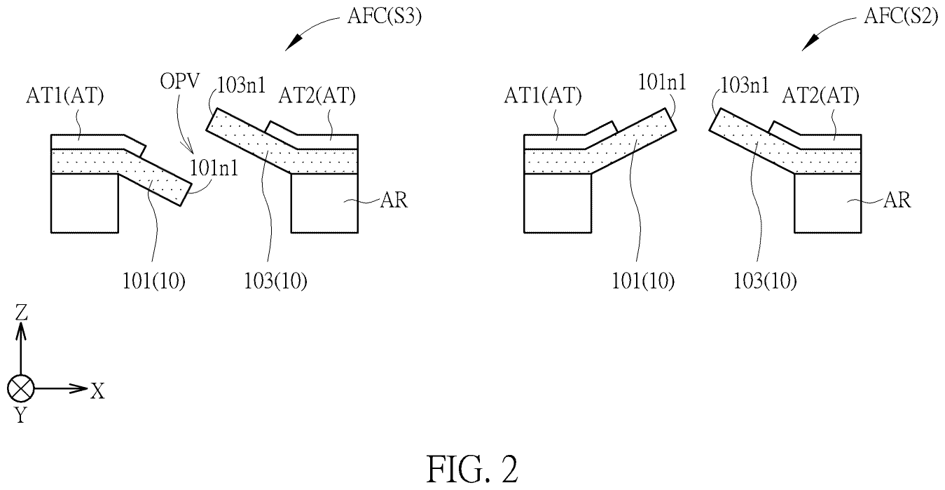

Referring to FIG. 1 and FIG. 2, FIG. 1 is a schematic diagram of a cross sectional view illustrating an air pump according to an embodiment of the present invention, and FIG. 2 is a schematic diagram of a cross sectional view illustrating a common mode movement and a differential mode movement of the air pump according to an embodiment of the present invention, wherein the air pump AFC shown in FIG. 1 is in an intermediate status S1. As shown in FIG. 1 and FIG. 2, the air pump AFC is configured to generate an airflow. In some embodiments, the air pump AFC may be configured to produce a plurality of air pulses, and the airflow may consist of the air pulses, wherein the air pump AFC may produce the air pulses at any suitable pulse rate. For example, the air pump AFC may produce the air pulses at an ultrasonic (pulse) rate higher than a maximum human audible frequency (e.g., 16 kHz, 20 kHz or 22 kHz), such that the user cannot hear the operation of the air pump AFC configured to generate the airflow and/or the air pulses, but not limited thereto.

As shown in FIG. 1, the air pump AFC may include at least one anchor structure AR and at least one film structure 10 anchored by/on the anchor structure AR, wherein the anchor structure AR may be disposed outside the film structure 10. The film structure 10 and the anchor structure AR may include any suitable material(s). In some embodiments, the film structure 10 and the anchor structure AR may individually include silicon (e.g., single crystalline silicon or poly-crystalline silicon), silicon compound (e.g., silicon carbide, silicon oxide), germanium, germanium compound, gallium, gallium compound (e.g., gallium nitride or gallium arsenide), other suitable material or a combination thereof, but not limited thereto. In some embodiments, the film structure 10 and the anchor structure AR may have the same material.

In the operation of the air pump AFC, the film structure 10 may be actuated to have a movement, and the anchor structure AR may be immobilized. Namely, the anchor structure AR may be a fixed end (or fixed edge) respecting the film structure 10 during the operation of the air pump AFC. In some embodiments, the film structure 10 may be actuated to move upwards and downwards, but not limited thereto. In the present invention, the terms “move upwards” and “move downwards” represent that the film structure 10 moves substantially along a direction Z. Moreover, “upwards” may refer to the direction Z (i.e., +Z direction) while “downwards” may refer to a direction opposite to the direction Z (i.e., −Z direction). Namely, an actuating direction of the film structure 10 is parallel to the direction Z. In an embodiment, the direction Z may be a vertical direction and/or a top-view direction.

As shown in FIG. 1, the film structure 10 of the air pump AFC includes at least one slit SL, and the film structure 10 is divided into a plurality of flaps (e.g., flaps 101 and 103) by the slit(s) SL (i.e., the flaps are separated from each other by the slit(s) SL, and the slit(s) SL may be boundaries of the flaps), wherein the number of the flaps may be designed based on requirement(s). For example, as shown in FIG. 1, the film structure 10 may be divided into a flap 101 and a flap 103 by the slit(s) SL, the flap 101 and the flap 103 may be disposed opposite to each other, and at least one slit SL may be between the flap 101 and the flap 103. Note that the flap 101 and the flap 103 opposite to each other may form a flap pair in the film structure 10.

In FIG. 1, each of the flaps 101 and 103 of the film structure 10 has at least one anchor edge (or anchor end) anchored on the anchor structure AR and at least one free edge (or free end) which is not permanently anchored on any component within the air pump AFC, and the anchor edge(s) and the free edge(s) of each of the flaps 101 and 103 may be designed based on requirement(s). For example (as shown in FIG. 1), the slit SL may define one free edge (e.g., a first free edge 101n1) of the flap 101 and one free edge (e.g., a second free edge 103n1) of the flap 103, this free edge (e.g., the first free edge 101n1) of the flap 101 may be opposite to the anchor edge of the flap 101, and this free edge (e.g., the second free edge 103n1) of the flap 103 may be opposite to the anchor edge of the flap 103, but not limited thereto.

In the present invention, the number of the slit(s) SL included in the film structure 10 may be adjusted based on requirement(s), and the slit(s) SL may be disposed at any suitable position of the film structure 10 and have any suitable top-view pattern. For example, the slit SL may be a straight slit, a curved slit, a combination of straight slits, a combination of curved slits or a combination of straight slit(s) and curved slit(s).

The air pump AFC may include an actuator AT configured to actuate the film structure 10 to generate the airflow and/or the air pulses, wherein the actuator AT may be disposed at any suitable position, and the position of the actuator AT may be related to the actuating method of the actuator AT. For instance, in FIG. 1, the actuator AT may overlap the film structure 10 in the direction Z, but not limited thereto. For instance, in FIG. 1, the actuator AT may be disposed on the film structure 10, but not limited thereto. For instance, in FIG. 1, the actuator AT may be in contact with the film structure 10, but not limited thereto. As shown in FIG. 1, the actuator AT may be divided into an actuator AT1 disposed on the flap 101 and an actuator AT2 disposed on the flap 103.

The actuator AT has a monotonic electromechanical converting function with respect to the movement of the film structure 10 along the direction Z. In some embodiments, the actuator AT may include a piezoelectric actuator, an electrostatic actuator, a nanoscopic-electrostatic-drive (NED) actuator, an electromagnetic actuator or any other suitable actuator, but not limited thereto. For example, in an embodiment, the actuator AT may include a piezoelectric actuator, the piezoelectric actuator may contain such as two electrodes and a piezoelectric material layer (e.g., lead zirconate titanate, PZT) disposed between the electrodes, wherein the piezoelectric material layer may actuate the film structure 10 based on driving signals (e.g., driving voltages and/or driving voltage difference between two electrodes) received by the electrodes, but not limited thereto. For example, in another embodiment, the actuator AT may include an electromagnetic actuator (such as a planar coil), wherein the electromagnetic actuator may actuate the film structure 10 based on a received driving signals (e.g., driving current) and a magnetic field (i.e. the film structure 10 may be actuated by the electromagnetic force), but not limited thereto. For example, in still another embodiment, the actuator AT may include an electrostatic actuator (such as conducting plate) or a NED actuator, wherein the electrostatic actuator or the NED actuator may actuate the film structure 10 based on a received driving signals (e.g., driving voltage) and an electrostatic field (i.e. the film structure 10 may be actuated by the electrostatic force), but not limited thereto. In the following, the actuator AT may be a piezoelectric actuator for example.

For example, if the air pump AFC is a MEMS chip, the film structure 10, the anchor structure AR and the actuator AT are MEMS structures in the MEMS chip, but not limited thereto. Furthermore, since the air pump AFC generates the airflow and/or the air pulses by actuating the film structure 10 through the actuator AT, the air pump AFC may be a bladeless fan, but not limited thereto.

In the present invention, the film structure 10 (the flaps 101 and 103) is actuated/controlled to move upwards and downwards by the actuator AT, such that a vent opening OPV related to the slit SL is formed/opened or closed (i.e., the film structure 10 is configured to form/open or close the vent opening OPV), wherein the vent opening OPV is formed between opposite sidewalls of the slit SL (i.e., the vent opening OPV is formed between the flap 101 and the flap 103). Namely, the vent opening OPV is formed because of the slit SL. In the condition “the vent opening OPV is closed/sealed”, the air is hard to flow to pass through a space between two opposite sidewalls of the slit SL, meaning that a flowing resistance of the vent opening OPV is large or larger than a threshold. In the condition “the vent opening OPV is formed/opened”, the air easily flows to pass through a space between two opposite sidewalls of the slit SL, meaning that the flowing resistance of the vent opening OPV is low or lower than another threshold.

In the present invention, the air pump AFC may generate the airflow and/or the air pulse by any suitable airflow producing method. For example, an airflow producing method related to FIG. 1 and FIG. 2 are described in the following, and this airflow producing method generates the airflow and/or the air pulse by changing the state of the vent opening OPV and changing the air pressures on two opposite sides of the film structure 10.

As shown in FIG. 1, in the intermediate status S1 of the air pump AFC, the film structure 10 (the flap pair) may be actuated and maintained as a first position which is substantially horizontal in the cross sectional view, and the vent opening OPV may be temporarily closed (or even temporarily sealed), such that the air may be hard to flow to pass through a space between two opposite sidewalls of the slit SL. In FIG. 1, two opposite sidewalls of the slit SL (i.e., the first free edge 101n1 of the flap 101 and the second free edge 103n1 of the flap 103) partially or fully overlap with each other in a horizontal direction (a gap of the slit SL is shown in FIG. 1), so as to make the vent opening OPV closed and have the larger flowing resistance. In an embodiment, the horizontal direction generally means a direction parallel to a horizontal plane, such as a direction X and a direction Y perpendicular to the direction Z.

In FIG. 1, since a size of the gap GP of the slit SL (or a width of the slit SL) should be sufficiently small, the airflow through the gap GP (i.e., a narrow channel) can be highly damped due to viscous forces/resistance along the walls of the airflow pathways, known as boundary layer effect within field of fluid mechanics. Accordingly, the airflow flowing through the gap GP in the intermediate status S1 is significantly small or negligible. In other words, when the air pump AFC is in the intermediate status S1, the vent opening OPV is closed and even sealed. The size of the gap GP of the slit SL (or a width of the slit SL) may be designed based on requirement(s). For instance, the size of the gap GP of the slit SL (or a width of the slit SL) may be less than or equal to 5 μm, less than or equal to 3 μm, or less than or equal to 2 μm, or may range from 1 μm to 2 μm, but not limited thereto. Note that the size of the vent opening OPV in the intermediate status S1 is equivalent to the size of the gap GP.

In FIG. 2, the film structure 10 (the flap pair) may be actuated to perform a common mode movement S2, such that the flap 101 and the flap 103 are simultaneously actuated to move toward the same direction. For example, the flap 101 and the flap 103 may be simultaneously actuated to move upwards or downwards along the direction Z. For example, in the end of the common mode movement S2, a distance between the flap 101 and the first position and a distance between the flap 103 and the first position are the same.

As shown in FIG. 2, when the film structure 10 (the flap pair) is actuated to perform the common mode movement S2, the vent opening OPV may be temporarily closed (or even temporarily sealed), such that the air may be hard to flow to pass through a space between two opposite sidewalls of the slit SL. In FIG. 2, two opposite sidewalls of the slit SL (i.e., the first free edge 101n1 of the flap 101 and the second free edge 103n1 of the flap 103) partially or fully overlap with each other in the horizontal direction, so as to make the vent opening OPV closed and have the larger flowing resistance.

When the film structure 10 (the flap pair) is actuated to perform the common mode movement S2, since the vent opening OPV is temporarily closed and has the larger flowing resistance, the air pressures on two opposite sides of the film structure 10 are different to cause an air-pressure difference. Namely, the film structure 10 (the flap pair) performs the common mode movement S2 to form an air pressure variation.

In FIG. 2, the film structure 10 (the flap pair) may be actuated to perform a differential mode movement S3, such that the flap 101 and the flap 103 are simultaneously actuated to move toward opposite directions. For example, the flap 101 may be actuated to move downwards and the flap 103 may be actuated to move upwards (as shown in FIG. 2), or the flap 101 may be actuated to move upwards and the flap 103 may be actuated to move downwards. For example, in the end of the differential mode movement S3, a distance between the flap 101 and the first position and a distance between the flap 103 and the first position are the same.

As shown in FIG. 2, when the film structure 10 (the flap pair) is actuated to perform the differential mode movement S3, the vent opening OPV may be temporarily opened, such that the air may easily flow to pass through a space between two opposite sidewalls of the slit SL. In FIG. 2, two opposite sidewalls of the slit SL (i.e., the first free edge 101n1 of the flap 101 and the second free edge 103n1 of the flap 103) do not overlap with each other in the horizontal direction, so as to make the vent opening OPV opened and have the lower flowing resistance.

When the film structure 10 (the flap pair) is actuated to perform the differential mode movement S3, if the air-pressure difference exists between two opposite sides of the film structure 10, the air naturally flows to pass through the vent opening OPV due to this air-pressure difference and the lower flowing resistance of the vent opening OPV, such that the airflow and/or the air pulse can be generated.

Accordingly, the airflow producing method of this embodiment may generate the airflow and/or the air pulse by actuating the film structure 10 (the flap pair) to perform the common mode movement S2 and the differential mode movement S3. For instance, one period of the airflow producing method of this embodiment may include four steps, but not limited thereto. The first step of the airflow producing method may be that the film structure 10 (the flap pair) is actuated to perform the common mode movement S2 to make the air-pressure difference exist between two opposite sides of the film structure 10. The second step of the airflow producing method may be that the film structure 10 (the flap pair) is actuated to recover the intermediate status S1. The third step of the airflow producing method may be that the film structure 10 (the flap pair) is actuated to perform the differential mode movement S3 to make air naturally flows to pass through the vent opening OPV due to this air-pressure difference and the lower flowing resistance of the vent opening OPV, such that the airflow and/or the air pulse can be generated. The fourth step of the airflow producing method may be that the film structure 10 (the flap pair) is actuated to recover the intermediate status S1. By repeating the periods of the airflow producing method of this embodiment, the air pulses may form the airflow continuously.

A frequency of the period may be designed based on the pulse rate of the air pulse, wherein the frequency of the period may be synchronous with the pulse rate of the air pulse. In the present invention, a frequency/rate is synchronous with another frequency/rate generally refers that this frequency/rate is this another frequency/rate times a rational number (i.e., N/M, wherein N and M represent integers). In some embodiments, the frequency of the period may be the same as the pulse rate of the air pulse. In some embodiments, the film structure 10 (the flap pair) performs the common mode movement S2 to form the air pressure variation with a pressure variant frequency synchronous with the frequency of the period, and the film structure 10 (the flap pair) performs the differential mode movement S3 to form the vent opening OPV at an opening rate synchronous with the pressure variant frequency and the frequency of the period. For instance, the frequency of the period, the pulse rate of the air pulse, the pressure variant frequency and the opening rate are the same. For instance, if the air pump AFC produces the air pulses at an ultrasonic rate, the pressure variant frequency and the opening rate are synchronous with this ultrasonic rate.

Flowing directions of the airflow and the air pulse are determined by a direction of the common mode movement S2 performed by the film structure 10 (the flap pair). When the film structure 10 (the flap pair) is actuated to move upwards (or downwards) for only performing one type of common mode movement S2 in the first step of the several periods, the types of the air-pressure differences in the first step of these periods are the same, thereby making the flowing directions of the air pulses generated in these periods (the third step) be the same. Thus, the air pump AFC shall produce single-ended (SE) air pulses or SE-liker air pulses. Also, the air pulse may be asymmetric.

In the present invention, a waveform of the SE air pulse or a waveform of the SE-liker air pulse may refer that the waveform is (substantially) unipolar with respect to certain level. For instance, the SE air pulse or the SE-liker air pulse may refer to the waveform which is (substantially) unipolar with respect to ambient pressure (e.g., 1 ATM). Namely, the SE air pulses or the SE-liker air pulses constitute a net air movement or a net airflow toward one single direction.

The airflow producing method of the present invention is not limited by the above. In one period of the airflow producing method, the number of the steps and the order of the actuating movements of the film structure 10 (the flap pair) may be designed based on requirement(s).

In another aspect, for any common mode movements S2 of the flap pair, a pair of acoustic pressure waves will be produced, one in space on a side of the film structure 10, and one in space on an opposite side of the film structure 10. These two acoustic pressure waves will be of the same magnitude but of opposite polarities. As a result, when the vent opening OPV is opened, the air-pressure difference between the two air volumes in the vicinity of the vent opening OPV would neutralize each other. Therefore, when the timing of differential mode movement S3 reaching its peak (i.e., the timing which the vent opening OPV is maximum) is aligned to the timing of acceleration of common mode movement S2 reaching its peak, the acoustic pressure supposed to be generated by the common mode movement S2 shall be subdued/eliminated due to the opening of the vent opening OPV, causing the auto-neutralization between two acoustic pressures on the two opposite sides of the film structure 10, where the two acoustic pressures would have same magnitude but opposite polarities. It means, when the vent opening OPV is opened, the air pump AFC would produce (near) net-zero air pressure. Therefore, when the opened period of the vent opening OPV overlaps a time period of one of the (two) polarities of acceleration of common mode movement S2 of the flap pair, the air pump AFC shall produce SE air pulses or SE-liker air pulses.

Furthermore, by aligning the timing of opening of the vent opening OPV to the timing of acceleration of common mode movement S2 of the flap pair, the air pump AFC would be able to produce asymmetric air pulses.

In some embodiments, the film structure 10 (the flap pair) may be actuated to perform the common mode movement S2 and the differential mode movement S3 simultaneously, but not limited thereto. In some embodiments, the film structure 10 may include other part to make the common mode movement S2 and the differential mode movement S3 be performed by the film structure 10 simultaneously, but not limited thereto.

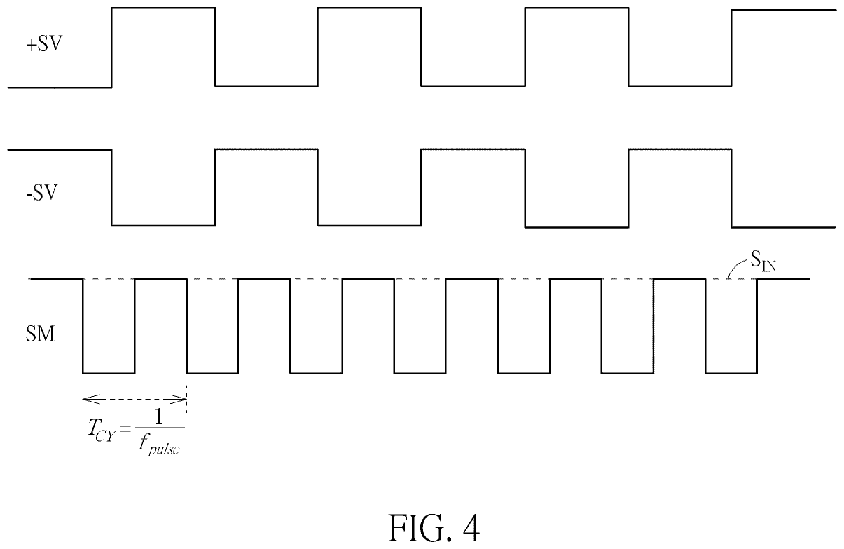

In the present invention, the actuator AT may receive any suitable signal to actuate the film structure 10. In some embodiments, the film structure 10 is actuated by a modulation-driving signal SM to perform the common mode movement S2 to form the air pressure variation, and the film structure 10 is actuated by a demodulation-driving signal SV to perform the differential mode movement S3 to form the vent opening OPV, wherein both the modulation-driving signal SM and the demodulation-driving signal SV are related to an output amplitude of the air pulse. Note that the demodulation-driving signal SV may be +SV or -SV shown in FIG. 4.

Furthermore, a modulation frequency of the modulation-driving signal SM and a demodulation frequency of the demodulation-driving signal SV are related to the pulse rate of the air pulse. For example, the modulation frequency and the demodulation frequency may be synchronous with the pulse rate of the air pulse, such that the modulation frequency and the demodulation frequency may be synchronous with the pressure variant frequency of the air pressure variation, the opening rate of the vent opening OPV and the frequency of the period, but not limited thereto.

In some embodiments, the actuator AT may receive the modulation-driving signal SM and the demodulation-driving signal SV at different times, but not limited thereto. In some embodiments, the actuator AT may include a plurality sub-parts in the top view, one sub-part may receive the modulation-driving signal SM, and another sub-part may receive the demodulation-driving signal SV, but not limited thereto. In some embodiments, the actuator AT may include a first electrode and a second electrode, the first electrode may receive the modulation-driving signal SM, and the second electrode may receive the demodulation-driving signal SV, but not limited thereto.

Furthermore, by controlling the modulation-driving signal SM and/or the demodulation-driving signal SV, the flowing direction of the airflow (the air pulse) produced by the air pump AFC may be reversible. Details of which may be referred to U.S. application Ser. No. 18/624,105, which are not narrated herein for brevity.

The details of the airflow generating MEMS device (which may be fabricated by semiconductor process), i.e., the air pump AFC (e.g., the structure, the driving signal and the movement) and their design/operational principles can be referred to U.S. Pat. No. 11,943,585, U.S. application Ser. Nos. 18/321,757 and 18/624,105 filed by same applicant. Thus, the contents of these US patents and US applications are incorporated herein by reference.

As mentioned earlier, the air pump AFC of the present application may be capable of producing asymmetric air pulses, and can be applied in cooling, drying, dehumidifying, heat dissipation, ventilation, air-sampling and/or air-pumping applications, where the (asymmetric) air pulses are produced to form a net air movement constantly in one direction.

Furthermore, the air pump AFC of the present invention for airflow applications may be disposed within an air quality sensing device, which is to sense, e.g., a density of specific particle(s) (e.g., PM 2.5 or PM 10 (PM: Particulate Matter)) or compound(s) (e.g., ozone (O₃), nitrogen dioxide (NO₂), sulfur dioxide (SO₂) and carbon monoxide (CO)) in the air. Hence, a size of the air quality sensing device may be significantly reduced.

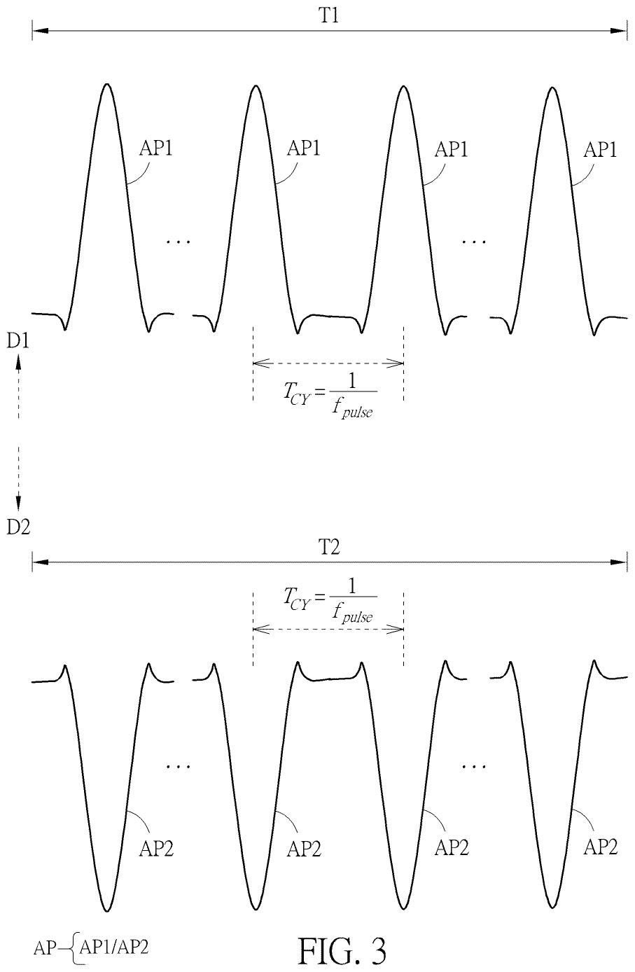

For example, FIG. 3 illustrates a schematic diagram of air pulses AP according to an embodiment of the present invention. The air pulses may be generated by the air pump AFC of present application, which comprises a film structure 10. As mentioned earlier, the film structure 10 of the air pump AFC may be actuated to perform a movement to generate the air pulses AP at an ultrasonic rate fₚᵤₗₛₑ (e.g., 96 KHz or 192 KHz), which may be a reciprocal of the operating cycle T_{CY} of the ultrasonic carrier frequency f_{UC} for example. In this case, the ultrasonic rate fₚᵤₗₛₑ may be the ultrasonic carrier frequency f_{UC}. The air pulses AP may produce a net airflow toward a single direction.

In an embodiment, first air pulses AP1 may produce a first net airflow constantly toward one single direction, e.g., a first direction D1. Taking FIG. 3 as an example, during the first period of time T1, the air pulses AP are all toward first direction D1. When the first time period T1 is at least or longer than a reciprocal of a minimum audible frequency, the first net airflow produced by the first air pulses AP1 may be considered as constantly toward one single direction D1. For example, for a minimum audible frequency being acknowledged as 10 Hz, when the first time period T1 is at least or longer than 0.1 second, the first net airflow may be considered as constantly toward one single direction D1. Note that, first amplitude(s), corresponding to the first air pulses AP1 toward the first direction D1, may or may not be the same.

On the other hand, the air pump AFC may produce second air pulses AP2, and the second air pulses AP2 may produce a second net airflow constantly toward a second direction D2, opposite to the first direction D1. In an embodiment, when the air pump AFC produces significant airflow or air movement and the air pulses toggling between the first direction D1 and the second direction D2 is not discernible, the first net airflow may be considered as constantly toward direction D1 during period T1, and/or the second net airflow may be considered as constantly toward direction D2 during period T2.

The film structure may be actuated by a demodulating-driving signal (e.g., ±SV) and a modulating-driving signal (e.g., SM). Note that, in the present application, SM may be referred to modulation signal, which is also a kind of driving signal. Similarly, ±SV may be referred to demodulation signal, which is also a kind of driving signal.

FIG. 4 illustrates schematic waveforms of the demodulation signals (±SV) and the modulation signal (SM) according to another embodiment of the present invention, neglecting transition between high/low voltages thereof. As shown in FIG. 4, the modulation/driving signal (SM) may be generated according to an input signal (e.g., a input audio signal S_{IN}), which comprises or is a (nonzero) direct current (DC) offset (e.g., a (nonzero) DC voltage). In other words, the input signal may be simply a DC signal, but not limited thereto.

In an embodiment, the DC offset may be related to the direction of the net airflow. For example, during a first period of time T1, the air pulses (AP) may produce a first net airflow constantly toward the first direction D1 in response to the DC offset being positive. On the other hand, during a second period of time T2, the air pulses generated by the air pump AFC may produce the second net airflow constantly toward the second direction D2, which is opposite to the first direction D1, in response to the DC offset being negative. In this regard, the air pump AFC or airflow generating device of the present invention may be viewed as a voltage-to-airflow converter, which can convert voltage into airflow.

In addition to polarity of the DC offset, the direction of net airflow may also be determined/controlled via phase between the modulation signal (SM) and the demodulation signal (±SV). For example, in FIG. 4, transitions of the demodulation signal (±SV) are aligned to interval of the modulation signal (SM) being low. In this case, the air pump AFC may produce airflow toward a third direction for example. When (phase of) the demodulation signal (±SV) is shifted such that transitions of the demodulation signal (±SV) are aligned to interval of the modulation signal (SM) being high, the air pump AFC would produce airflow toward a fourth direction opposite to the third direction. In short, the direction of the net airflow produce by the air pump AFC may be determined/controlled via phase (difference) between the modulation signal (SM) and the demodulation signal (±SV).

The strength/volume of a net airflow may be related to or a function of the magnitude of the DC offset. By maintaining an airflow direction (either the first direction or the second direction), the air pump AFC is able to dissipate heat, dehumidify, provide ventilation, provide air-sampling application, provide air-pumping application and/or facilitate air circulation. In this case, the air pump AFC can be regarded as a bladeless fan. That is, the air pump AFC may also be regarded as bladeless fan, especially when the driving signal or modulation-driving signal applied thereto is generated according to an input signal comprising nonzero DC component/offset. In the present invention, the terms of air-pulse generating device, airflow generating device, air pump and blower may be used interchangeably.

Due to the small size of the air pump of the present invention, it is possible for an air quality sensing module comprising the air pump to be disposed within (integrated into) a handheld device. For example, the handheld device may be a (smart) phone, a (smart) watch or other suitable handheld portable device.

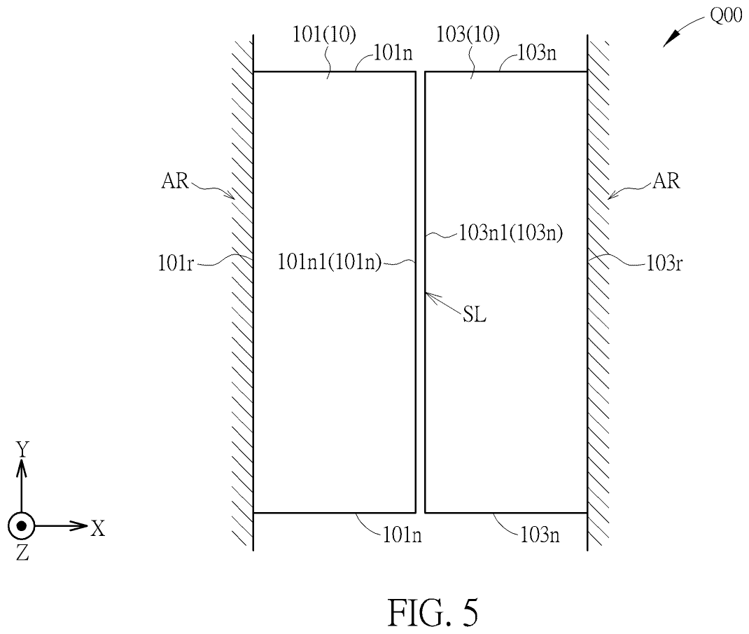

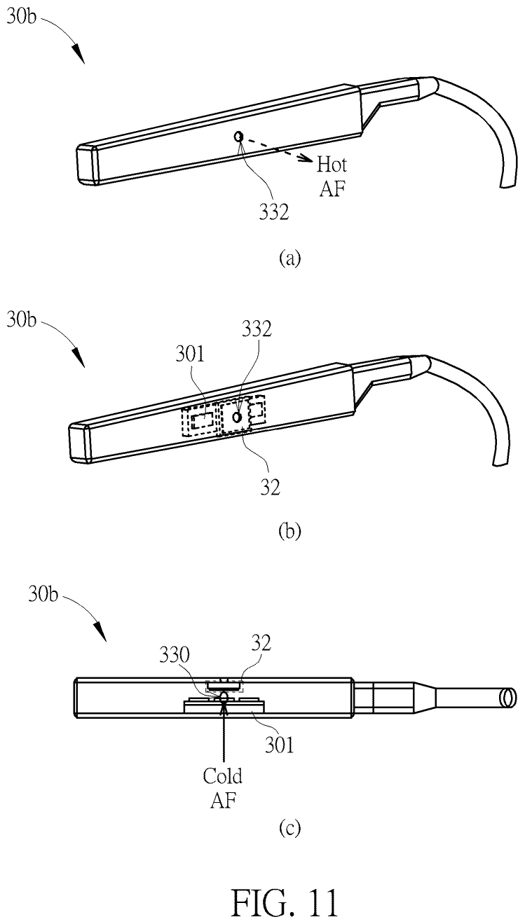

Referring to FIG. 5, FIG. 5 is a schematic diagram of an air pump or an airflow generating device according to an embodiment of the present invention. Note that the airflow generating device Q00 shown in FIG. 5 may be an example used in the air quality sensing module, and the airflow generating device Q00 may be a MEMS chip for example. As shown in FIG. 11, two flaps 101 and 103 of the airflow generating device Q00 are opposite to each other in a top view viewing along a top-view direction (i.e., the direction Z), and the actuators AT1 and AT2 are respectively disposed on the flaps 101 and 103. Note that the two flaps 101 and 103 form a flap pair.

In FIG. 5, the flap 101 includes a first anchored edge 101r anchored on the anchor structure AR, and the flap 101 includes first free edges 101n other than the first anchored edge 101r which are non-anchored. Similarly, the flap 103 includes a second anchored edge 103r anchored on the anchor structure AR, and the flap 101 includes second free edges 103n other than the second anchored edge 103r which are non-anchored. Namely, each of the flaps 101 and 103 only has one anchored edge, and other edges are free edges.

In FIG. 5, a slit SL is formed between the flaps 101 and 103, such that the flaps 101 and 103 are divided by the slit SL, and one first free edge 101n1 of the flap 101 and one second free edge 103n1 of the flap 103 are defined by the slit SL (the first free edge 101n1 and the second free edge 103n1 are opposite sidewalls of the slit SL). Note that the vent opening OPV formed between the flaps 101 and 103 is formed because of the slit SL.

In short, the airflow generating device Q00 comprises two flaps disposed opposite to each other, and each flap has one edge anchored and the rest edges non-anchored. Simulation and experiments results validate that the airflow generating device of the present invention (e.g., Q00) is able to produce significant airflow. In some experiments, the airflow generating device of the present invention (e.g., Q00) may have PQ (Pressure-Airflow) curve with maximum (static/back) pressure Pₘₐₓ as 800˜1300 Pa and maximum airflow volume Qₘₐₓ as 25˜45 cc/sec, where Pa represents Pascal cc/sec represents cubic centimeters per second.

The airflow generating device of the present invention (e.g., Q00) may be disposed within various electronic devices. For example, the airflow generating device of the present invention may be disposed within a phone.

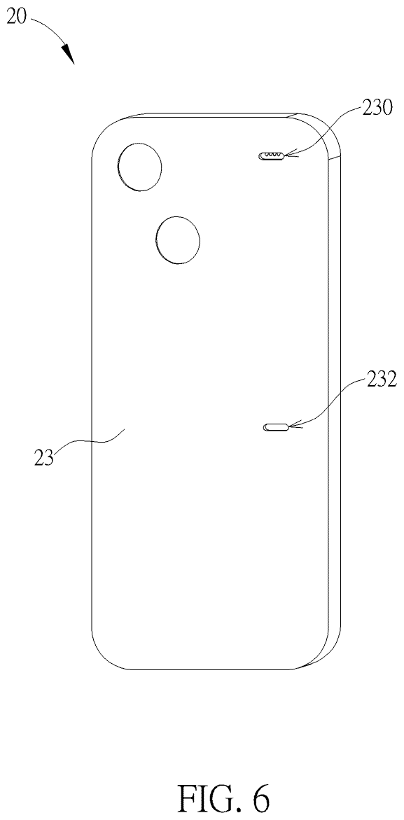

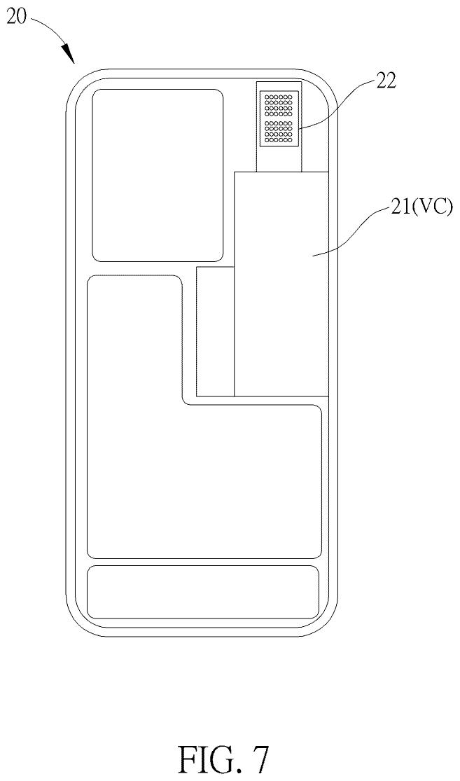

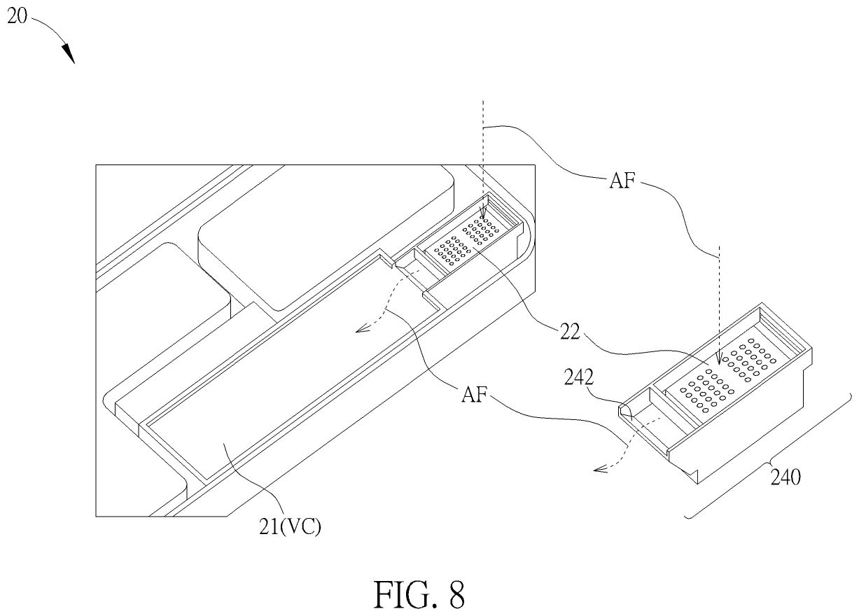

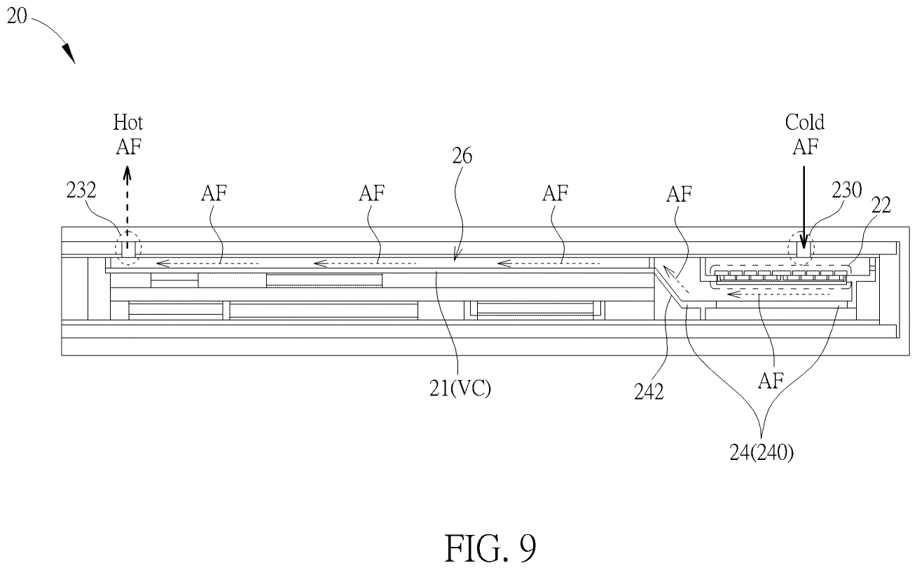

FIG. 6 is a schematic diagram of an appearance of a phone 20 according to an embodiment of the present invention. FIG. 7 is an internal layout diagram of the phone 20 according to an embodiment of the present invention. FIG. 8 is a (local) detailed view of the internal layout diagram of the phone 20 according to an embodiment of the present invention. FIG. 9 is a (local) cross-sectional view of the phone 20 according to an embodiment of the present invention.

The phone 20 comprises a back cover 23. An inlet 230 and an outlet 232 are formed on the back cover 23. A cold airflow (denoted as “cold AF” afterward) may flow into the phone 20 via the inlet 230 and a hot/warm airflow (denoted as “hot AF” afterward) may flow outward from the phone 20. The inlet 230 and the outlet 232 are connected to a flow channel (e.g., 26 shown later) formed within the phone 20.

The phone 20 comprises an airflow generating device 22 and a heat spreading component 21. The airflow generating device 22 may have the same or similar structure as the airflow generating device Q00. In an embodiment, the heat spreading component 21 may be or comprise a vapor chamber VC. The phone 20 may comprise (high performance) computing device as heat generating device. The (high performance) computing device as heat generating device may be attached to the vapor chamber VC such that the vapor chamber VC may dissipate heat generated by the computing device. The airflow generating device 20 may dissipate/remove heat from the vapor chamber VC or the heat spreading component 21.

The phone 20 may comprise a flow guide 24. The airflow generating device 20 may generate an airflow AF flowing through the flow guide 24. A flow channel 26 may be formed because of the flow guide 24. The flow guide 24 may guide or direct the airflow AF toward or through the flow channel 26.

The flow guide 24 may comprise a sub-guiding structure 240. The sub-guiding structure 240 may comprise a space or a cavity to accommodate the airflow generating device 20. The sub-guiding structure 240 may have function similar to a manifold. In an embodiment, the sub-guiding structure 240 may collect the airflow AF generated by the airflow generating device 22 and to direct the airflow AF toward or through the flow channel 26. In an embodiment, the sub-guiding structure 240 may comprise a ramped section 242 to guide/direct the airflow AF toward the flow channel 26.

The airflow generating device of the present invention may be disposed within a smart glasses. The smart glasses of the present invention refer to glasses equipped with computation capability or computing chips. As the computation load is increasing recently, especially the coming AI or edge AI (AI: artificial intelligence) era, the computing chips may generate uncomfortable heat. It is necessary to dissipate heat generated by the computing chips. The airflow generating device of the present invention, having compact size and capable of generating airflow, is suitable for integration within smart glasses.

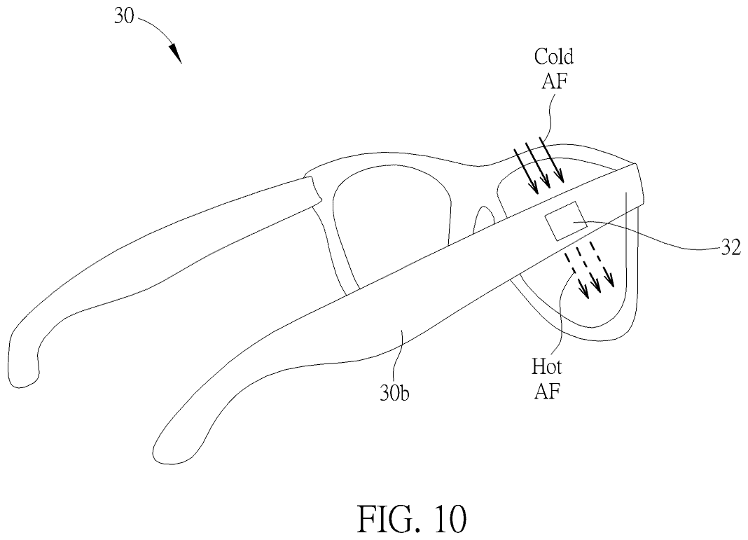

FIG. 10 is a schematic diagram of a smart glasses 30 according to an embodiment of the present invention. FIG. 11 is a schematic diagram of a temple structure 30b of the smart glasses 30 according to an embodiment of the present invention. FIG. 11(a) is a schematic diagram of an appearance of the temple structure 30b according to an embodiment of the present invention. FIG. 11(b) is a schematic diagram of a transparent view of the temple structure 30b. FIG. 11(c) is a schematic diagram of a bottom view of the temple structure 30b.

The smart glasses 30 may comprise the temple structure 30b and an airflow generating device 32. The airflow generating device 32 may have the same or similar structure as the airflow generating device Q00. The airflow generating device 30 may be disposed within the temple structure 30b of the smart glasses 30. Within the temple structure 30b, the airflow generating device 32 and heat generating component(s) 301 are disposed, where 301 may be computing chip or any kind of processor or processing unit.

The smart glasses 30 may also comprise an inlet 330 and an outlet 332. The temple structure 30b shown in FIG. 11 may be a left arm of the smart glasses 30. The outlet 332 faces outward or is positioned on an outer side of the temple structure, and the hot airflow (denoted as hot AF) flows away from the user or away from the outlet 332 on the outer side of the temple structure 30b. The inlet 330 may be positioned either on a top side or on a bottom side of the temple structure 30b, in general. In the embodiment shown in FIG. 11(c), the inlet 330 is positioned on the bottom side of the temple structure 30b, and the cold airflow (denoted as cold AF) flows from the bottom of the temple structure 30b. In this regard, user hardly feels the heat dissipating airflow (either cold or hot AF) and would not feel uncomfortable heat from the heat generating component(s) 301.

The airflow generating device of the present invention (e.g., Q00) may be disposed within a memory module.

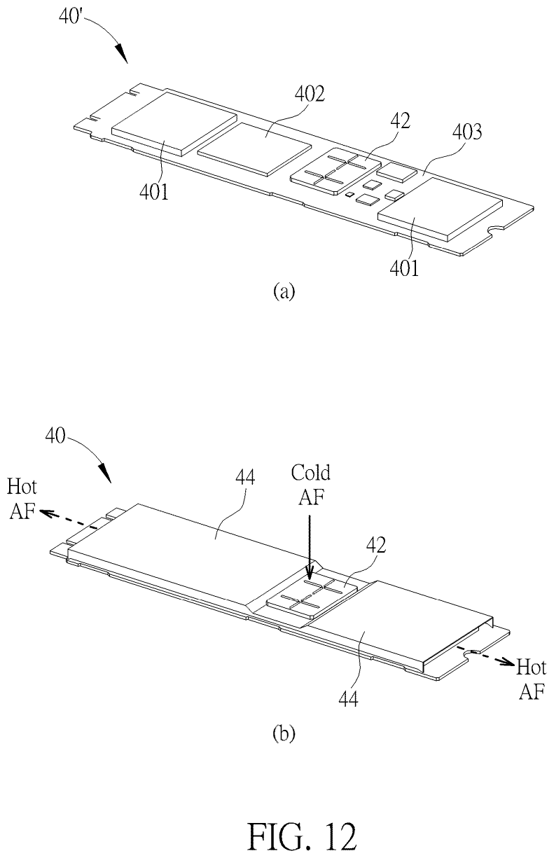

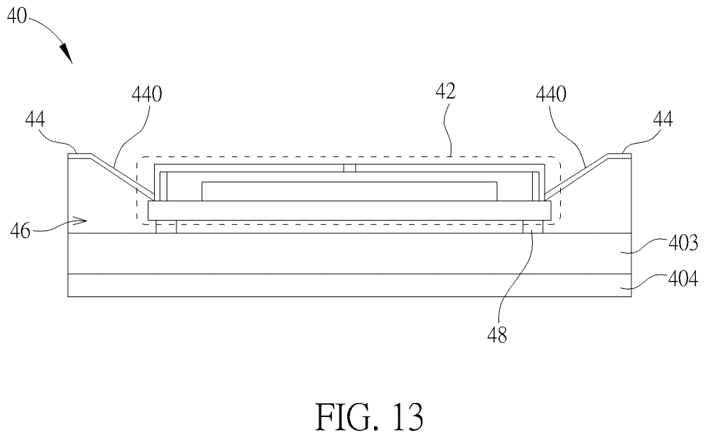

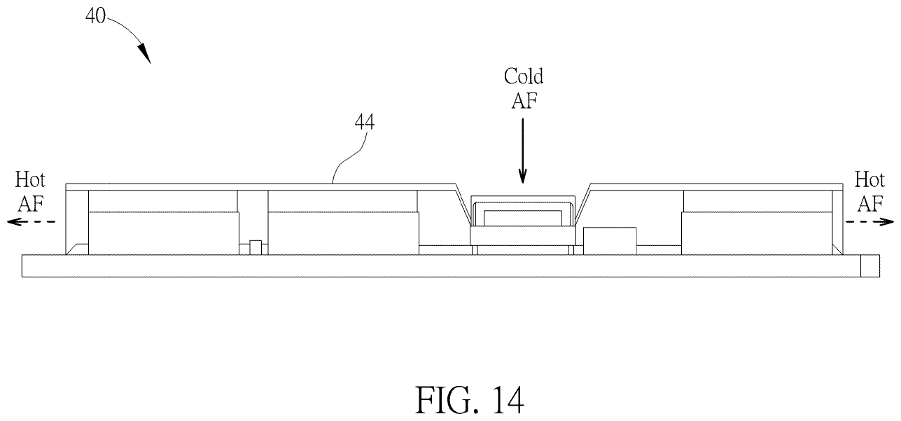

FIG. 12 is a schematic diagram of a memory module 40 and a memory module 40′ according to an embodiment of the present invention. FIG. 13 is a (local) cross-sectional view of the memory module 40 according to an embodiment of the present invention. FIG. 14 is a cross-sectional view of the memory module 40 according to an embodiment of the present invention.

The memory module 40′ may be an SSD (SSD: Solid State Drive), which comprises memory chips (e.g., NAND Flash Memory) 401, a controller 402 and an airflow generating device 42. The airflow generating device 42 may have the same or similar structure as the airflow generating device Q00. The airflow generating device 42 may be disposed on or over a circuit board 403. In addition to the memory module 40′, the memory module 40 further comprises a cover or a flow guide 44. A flow channel 46 may be formed within or under the flow guide 44.

In addition, the memory module 40 may comprise a spacing structure 48, disposed between the airflow generating device 42 and the circuit board 403. The spacing structure 48 may be configured to form a gap between the airflow generating device 42 and the circuit board 403, such that an airflow AF generated by the airflow generating device 42 may flow through the gap formed because of the spacing structure 48, either toward or from the flow channel 46. Optionally, a TIM (TIM: Thermal Interface Material) 404 may be included and disposed under the circuit board 403, which is not limited thereto.

The memory chips 401 and the controller 402 may be considered as integrated circuits, which may generate heat when operating. The memory chips 401 and the controller 402 may be disposed under the flow guide 44 and encompassed by the flow channel 46, such that the heat generated by the memory chips 401 and the controller 402 may be dissipated through the airflow AF generated by the airflow generating device 42.

Furthermore, the flow guide 44 comprises a ramped section 440. The ramped section 440 may be configured to guide or direct the airflow AF from the airflow generating device 42 toward a lateral edge of the flow guide 44.

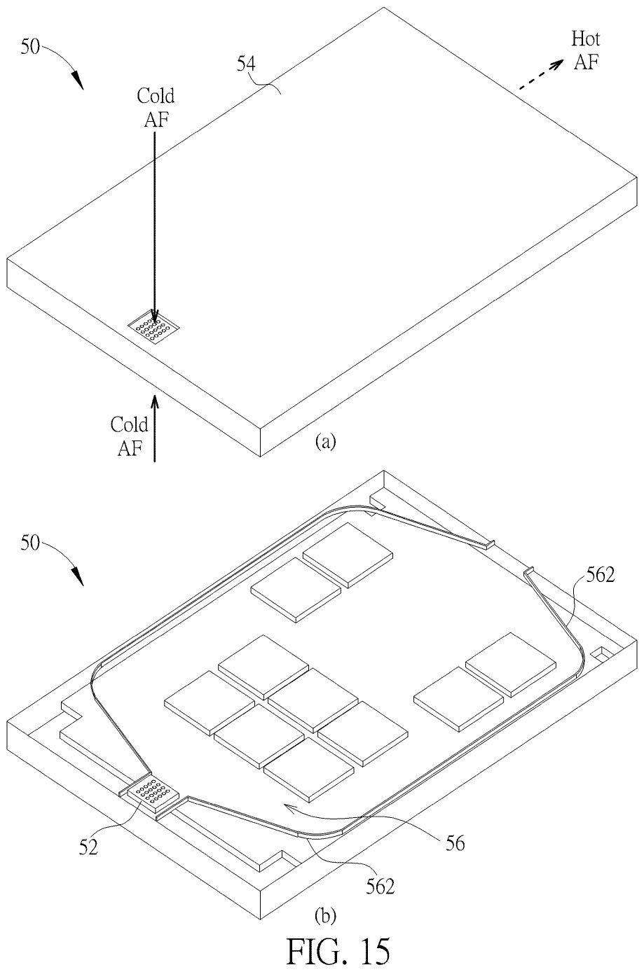

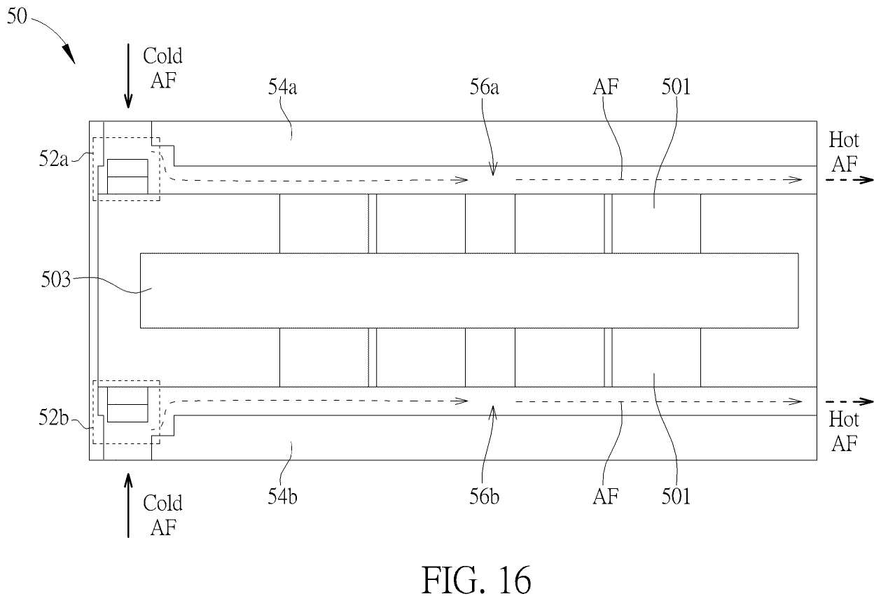

FIG. 15(a) is a schematic diagram of an appearance of a memory module 50 according to an embodiment of the present invention. FIG. 15(b) is a schematic diagram of a transparent view of the memory module 50. FIG. 16 is a schematic diagram of a cross-sectional view of the memory module 50.

The memory module 50 comprises a covering structure 54. A recessed area or a cavity may be formed within the covering structure 54 as a flow channel 56, where the recessed area or the cavity may be considered as a flow guide, which also denoted as “54”. Edges or boundaries 562 of the recessed area or the cavity, as the flow channel 56, are also shown in FIG. 15(b). As can be seen from FIG. 15(b), a width of the flow channel 56 increases from a lateral edge toward a central of the flow guide 54 or flow channel 56.

Specifically, the memory module 50 may have double-stacked structure, shown as FIG. 16. The memory module 50 may comprise airflow generating devices 52a, 52b, covering structures 54a, 54b. The airflow generating devices 52, 52a, 52b may have the same or similar structure as the airflow generating device Q00. Flow channels 56a, 56b are formed within the covering structures 54a, 54b. A circuit board 503 is included within the memory module 50, and integrated circuits 501 may be disposed either on a first (top) side or on a second (bottom) side of the circuit board 503.

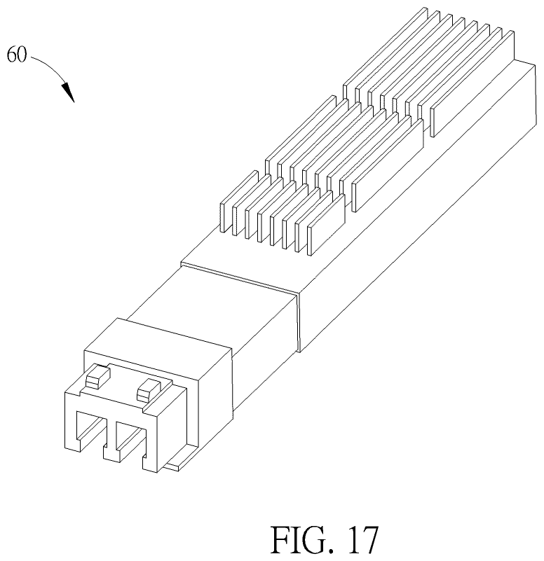

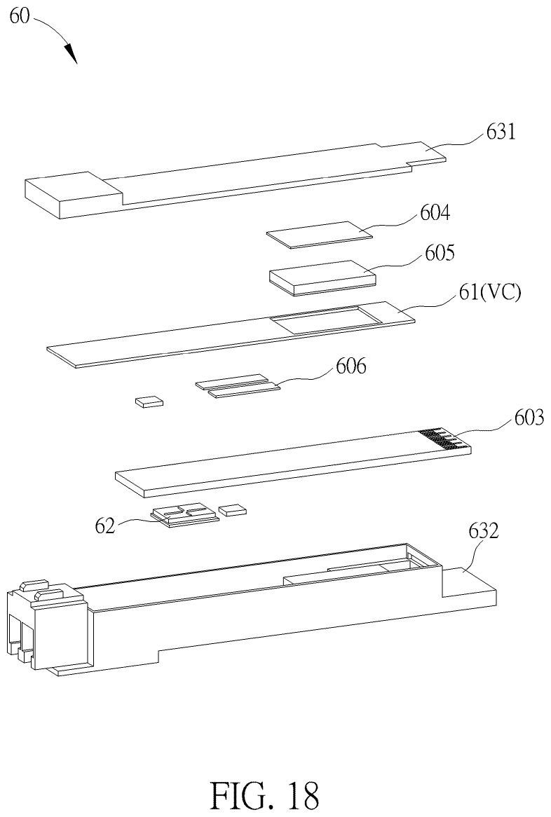

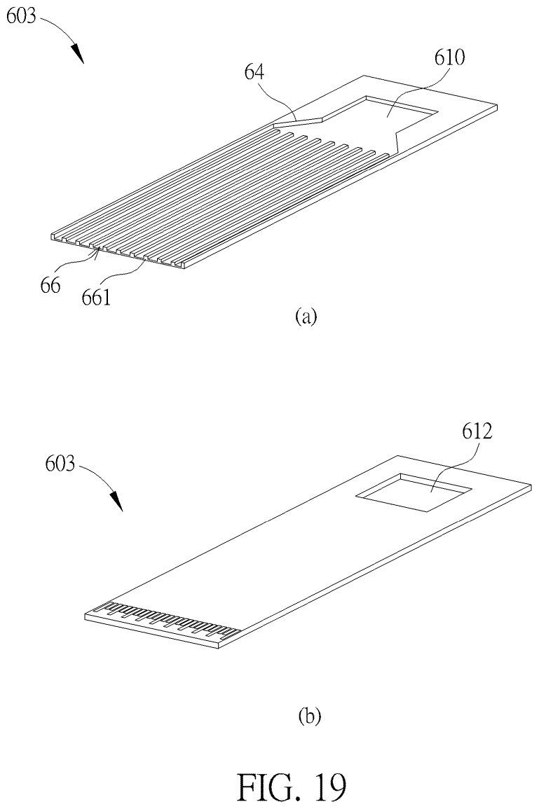

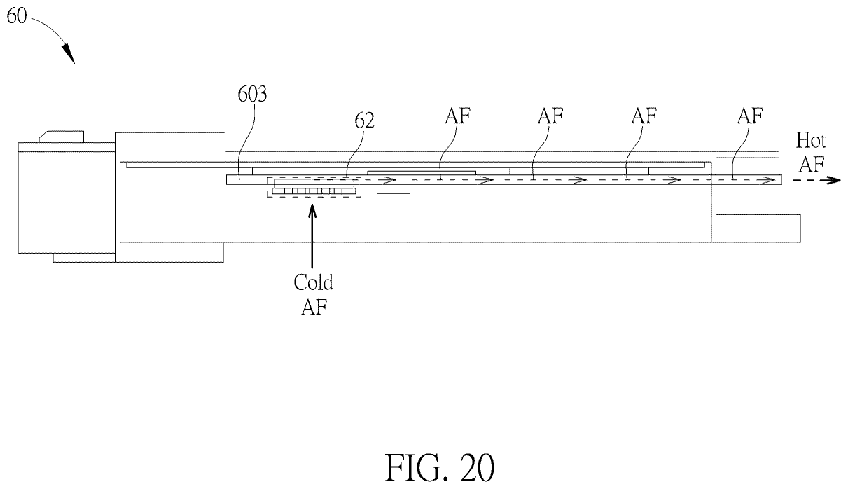

FIG. 17 is a schematic diagram of an appearance of a pluggable optical transceiver module 60 according to an embodiment of the present invention. FIG. 18 is a schematic diagram of an exploded view of the pluggable optical transceiver module 60. FIG. 19 is a schematic diagram of an appearance of a circuit board (e.g., 603) disposed within the pluggable optical transceiver module 60. FIG. 20 is a schematic diagram of a cross-sectional view of the pluggable optical transceiver module 60.

In the embodiments shown in FIGS. 17-20, the pluggable optical transceiver module 60 may be a QSFP (QSFP: Quad Small Form-factor Pluggable), which may be disposed within a server and the server may be disposed within a data center. The pluggable optical transceiver module 60 may comprise a first cover 631, a second cover 632, a TIM 604, a DSP (DSP: Digital Signal Processor) 605, a heat spreading component 61 (which may be or comprise a vapor chamber VC), integrated circuits 606, a circuit board 603 and an airflow generating device 62. The airflow generating device 62 may have the same or similar structure as the airflow generating device Q00.

The circuit board 603, which may be PCB (PCB: printed circuit board), may be formed to have flow channels (e.g., flow channels 66 shown in FIG. 19(a)). Within the flow channels 66, fin-type structures 661 may be formed on a first side of the circuit board 603. In addition, slots 610 and 612 may be formed on the first side and a second side of the circuit board. The slot 610 may be used to accommodate the airflow generating device 62. Therefore, the airflow AF generated by airflow generating device 62 may flow through the flow channels 66 and the fin-type structures 661, in order to facilitate heat dissipation.

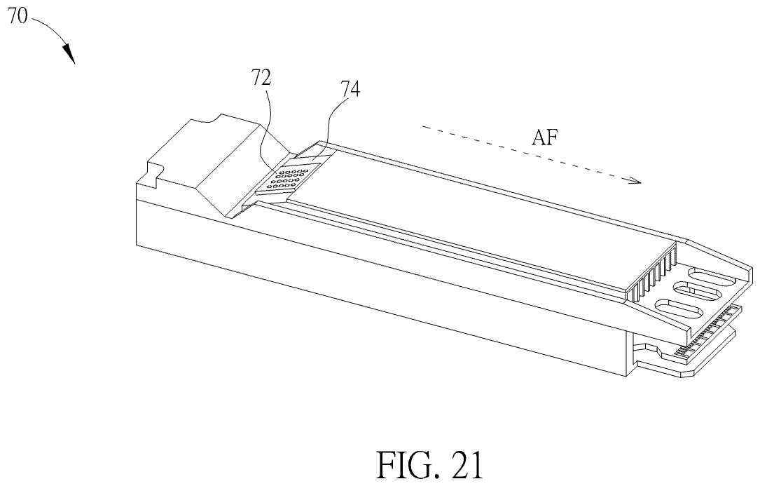

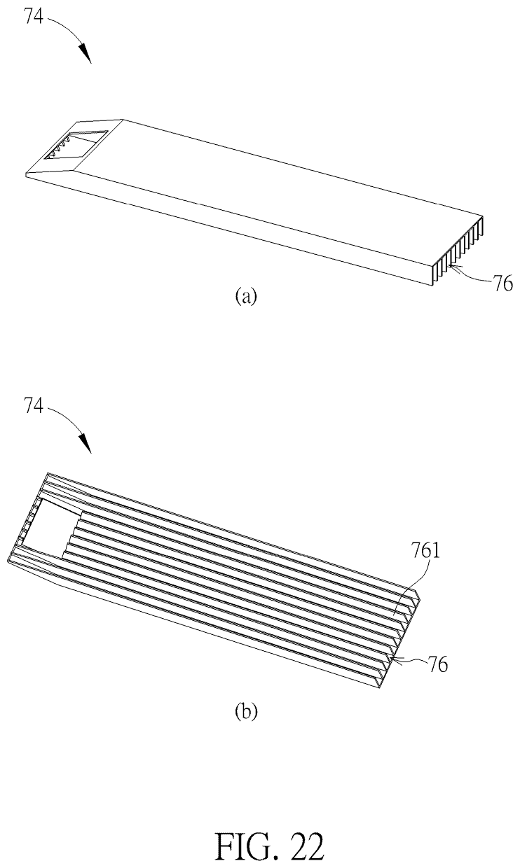

FIG. 21 is a schematic diagram of an appearance of a pluggable optical transceiver module 70 according to an embodiment of the present invention. FIG. 22 is a schematic diagram of an appearance of a cover or a flow guide (e.g., 74) disposed within the pluggable optical transceiver module 70.

In the embodiments shown in FIGS. 21-22, the pluggable optical transceiver module 70 may be an OSFP (OSFP: Octal Small Form Factor Pluggable), which may be disposed within a server and the server may be disposed within a data center. The pluggable optical transceiver module 70 may comprise an airflow generating device 72. The airflow generating device 72 may have the same or similar structure as the airflow generating device Q00.

The pluggable optical transceiver module 70 may also comprise a cover or a flow guide 74. The flow guide 74 may have fin-type structures 761, and flow channels 76 are formed between the fin-type structures 761. Therefore, the airflow AF generated by airflow generating device 72 may flow through the flow channels 76 and the fin-type structures 761, in order to facilitate heat dissipation.

In the present invention, phones, smart glasses, memory modules, pluggable optical transceiver modules may be considered as electronic devices, which is not limited thereto. Other kinds of electronic devices having airflow generating device(s) and flow channel(s) may be considered as being within the scope of the present invention.

In conclusion, the present invention discloses a high-performance airflow generating device specifically designed for integration into compact electronics. By utilizing a MEMS-based piezoelectric architecture, the device provides precise, localized cooling or air sampling through strategically designed flow channels and flow guides. Its thin profile allows it to be disposed within constrained environments such as smart glasses, SSD modules, and pluggable transceivers. Ultimately, this technology effectively manages the thermal energy from critical components like controllers and NAND chips, ensuring system reliability and enhanced performance in next-generation handheld and enterprise hardware.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An airflow generating device, comprising:
a first flap and a second flap, wherein the first flap and the second flap are opposite to each other in a top view viewing along a top-view direction;
a first actuator disposed on the first flap and a second actuator disposed on the second flap; and
an anchor structure;
wherein the first flap comprises a first anchored edge anchored on the anchor structure, and the first flap comprises first free edges other than the first anchored edge which are non-anchored;
wherein the second flap comprises a second anchored edge anchored on the anchor structure, and the second flap comprises second free edges other than the second anchored edge which are non-anchored.

2. A phone, comprising:
the airflow generating device of claim 1.

3. The phone of claim 2, comprising:
a flow guide configured to guide or direct an airflow toward or through a flow channel;
wherein the airflow is generated by the airflow generating device and flows through the flow channel.

4. The phone of claim 3,
wherein the flow guide comprises a sub-guiding structure.

5. The phone of claim 4,
wherein the sub-guiding structure comprises a ramped section;
wherein the ramped section is configured to guide or direct the airflow toward the flow channel.

6. The phone of claim 3, comprising:
a heat spreading component, disposed by the flow channel;
wherein the airflow flows through the flow channel and removes a heat away from the heat spreading component.

7. The phone of claim 6,
wherein the heat spreading component comprises a vapor chamber.

8. The phone of claim 2, comprising:
a back cover;
wherein an inlet and an outlet are formed on the back cover.

9. The phone of claim 8,
wherein the inlet and the outlet are connected to a flow channel formed within the phone.

10. A smart glasses, comprising:
a temple structure; and
the airflow generating device of claim 1, disposed within the temple structure.

11. The smart glasses of claim 10, comprising:
an outlet, positioned on an outer side of the temple structure;
wherein the airflow generating device generates an airflow flowing away from the outlet on the outer side of the temple structure.

12. The smart glasses of claim 10, comprising:
an inlet, positioned on a top side or a bottom side of the temple structure;
wherein the airflow generating device generates an airflow flowing toward the inlet on the top side or the bottom side of the temple structure.

13. A memory module, comprising:
the airflow generating device of claim 1.

14. The memory module of claim 13,
wherein the airflow generating device is disposed on or over a circuit board.

15. The memory module of claim 14, comprising:
a spacing structure, disposed between the airflow generating device and the circuit board;
wherein a gap is formed between the airflow generating device and the circuit board;
wherein the airflow generating device generates an airflow flowing through the gap.

16. The memory module of claim 13, comprising:
a flow guide;
wherein a flow channel is formed because of the flow guide.

17. The memory module of claim 16,
wherein the flow channel is formed between the flow guide and a circuit board;
wherein the flow guide is configured to guide or direct an airflow toward or through the flow channel.

18. The memory module of claim 16, comprising:
integrated circuits, disposed under the flow guide and encompassed by the flow channel.

19. The memory module of claim 16,
wherein the flow guide comprises a ramped section;
wherein the ramped section is configured to guide or direct an airflow from the airflow generating device toward a lateral edge of the flow guide.

20. The memory module of claim 16, comprising:
a covering structure;
wherein the flow guide is formed within the covering structure.

21. The memory module of claim 16,
wherein a width of the flow channel increases from a lateral edge toward a central of the flow guide;
wherein the airflow generating device is disposed by a first lateral edge of the flow guide, and a port is positioned adjacent to a second lateral edge of the flow guide.

22. A pluggable optical transceiver module, comprising:
the airflow generating device of claim 1.

23. The pluggable optical transceiver module of claim 22, comprising:
a flow guide;
wherein a flow channel is formed because of the flow guide.

24. An electronic device, comprising:
the airflow generating device of claim 1;
wherein a flow channel is formed within the electronic device.

25. The electronic device of claim 24, comprising:
fin-type structures;
wherein an airflow generated by the airflow generating device flows through the flow channel and the fin-type structures.

26. A smart glasses, comprising:
a temple structure; and
an airflow generating device, configure to generate an airflow, disposed within the temple structure.

27. A memory module, comprising:
an airflow generating device; and
a flow guide;
wherein a flow channel is formed because of the flow guide.

28. The memory module of claim 27, comprising:
a spacing structure, disposed between the airflow generating device and a circuit board;
wherein the airflow generating device is disposed on or over the circuit board;
wherein a gap is formed between the airflow generating device and the circuit board;
wherein the airflow generating device generates an airflow flowing through the gap;
wherein the flow channel is formed between the flow guide and the circuit board;
wherein the flow guide is configured to guide or direct the airflow toward or through the flow channel.

29. The memory module of claim 27,
wherein the flow guide comprises a ramped section;
wherein the ramped section is configured to guide or direct an airflow from a first section to a second section.

30. The memory module of claim 27, comprising:
a covering structure;
wherein the flow guide is formed within the covering structure;
wherein a width of the flow channel increases from a lateral edge toward a central of the flow guide.
